SEQUENCE LISTING (8 sequences)

SEQ ID NO: 1 - DNA, 111 bp, Homo sapiens
atgactgaatataaacttgtggtagttggagctggtggcgtaggcaagagtgccttgacgatacagctaattcagaatcattttgtggacgaatatgatccaacaatagag

SEQ ID NO: 2 - DNA, 34 bp, Artificial Sequence
  Synthetic KRAS probe
cgcgcgctcttgcctacgccaccagctccgcgcg

SEQ ID NO: 3 - DNA, 22 bp, artificial sequence
  Synthetic miR21
tagcttatcagactgatgttga

SEQ ID NO: 4 - DNA, 32 bp, artificial sequence
  Synthetic miR21 probe
gcgcgtcaacatcagtctgataagctacgcgc

SEQ ID NO: 5 - DNA, 92 bp, Artificial Sequence
  Synthetic target
aaaaaaaaaaaaaaaaaaaaaaaaaaaaaaaaaaatagcttatcagactgatgttgaaaaaaaaaaaaaaaaaaaaaaaaaaaaaaaaaaaa

SEQ ID NO: 6 - DNA, 22 bp, Artificial Sequence
  Synthetic probe
tcaacatcagtctgataagcta

SEQ ID NO: 7 - DNA, 92 bp, Artificial Sequence; identical to SEQ ID NO: 5
  Synthetic target

SEQ ID NO: 8 - DNA, 22 bp, Artificial Sequence
  Synthetic probe
tcaacatcagtctgataagcta

(19) United States
(12) Patent Application Publication
(10) Pub. No.: US 2020/0114350 A1
(43) Pub. Date: Apr. 16, 2020

(54) METHOD FOR NUCLEIC ACID DETECTION

(71) Applicants: Aurélien BANCAUD, Toulouse (FR); Rémi MALBEC, Toulouse (FR); Bayan CHAMI, Toulouse (FR); Inga NELYTE, Toulouse (FR)

(72) Inventors: Aurélien BANCAUD, Toulouse (FR); Rémi MALBEC, Toulouse (FR); Bayan CHAMI, Toulouse (FR); Inga NELYTE, Toulouse (FR)

(21) Appl. No.: 16/627,453

(22) PCT Filed: Jun. 29, 2018
(86) PCT No.: PCT/FR2018/051624

§ 371 Date: Dec. 30, 2019

(30) Foreign Application Priority Data
Jun. 30, 2017 (FR) 1756234

(51) Int. Cl. B01L 3/00; C12Q 1/6816

(52) U.S. Cl. CPC B01L 3/50273; B01L 2400/0421; C12Q 1/6816

(57) ABSTRACT

The object of the present invention is a method for detection of a nucleic acid in a sample by nucleic acid separation and enrichment by using a bidirectional electro-hydrodynamic flow in a non-newtonian liquid medium, where the method further comprises the addition of probes intended to hybridize with a target nucleic acid so as to introduce a modification of the molecular weight of the target nucleic acid during hybridization with the probes in order to allow the separation of nucleic acid/probe complexes from the nucleic acid alone or the probes alone when the sample is subject to hydrodynamic and electrical action in order to allow the detection of the target nucleic acid.

### FIELD OF THE INVENTION

The present invention relates to a method for detection of a nucleic acid, DNA or RNA, by separation and enrichment of nucleic acids by using a bidirectional electro-hydrodynamic flow in a non-newtonian liquid medium.

### DESCRIPTION OF THE RELATED ART

Detection of nucleic acid sequences is fundamental in a wide range of biological applications such as in vitro diagnostics, clinical diagnostics, research, etc. In particular, the demonstration of the presence of a specific nucleic acid sequence in a physiological sample constitutes the first line of development of diagnostic methods.

In the prior art various methods and devices are known with which to detect nucleic acids; these techniques rest on the detection of a hybrid molecule composed of the target nucleic acid molecule and a specific marked probe.

As an example, consider the recognition of DNA by molecular beacon, a tried-and-true method with which to detect point mutations on a 15 to 20 base fragment. However, this technique has low sensitivity, because the beacon is never completely extinguished in the absence of the target, so much so that the background noise can easily be a parasite signal when the target/beacon proportion is not correctly adjusted.

In fact, one of the crucial aspects in any nucleic acid detection method is the sensitivity.

The most used methods for nucleic acid detection are based on polymerase chain reaction (PCR). Real-time PCR, for example, is used for simultaneously amplifying and quantifying a targeted DNA molecule. PCR can also be applied to RNA amplification in a process called reverse transcriptase PCR (RT-PCR). RT-PCR is similar to regular PCR, with the addition of a step in which DNA is synthesized from the target RNA by using an enzyme called reverse transcriptase. A wide variety of RNA molecules have been used in RT-PCR, including ribosomal RNA, messenger RNA and genomic viral RNA.

Amplification of the target sequence in these PCR-based techniques takes time, increases the probability of errors and is very susceptible to contamination.

There is therefore a need to develop a high sensitivity nucleic acid detection method which is simple and quick to use.

### BRIEF SUMMARY OF THE INVENTION

Thus, the object of the present invention is a method for detection of a nucleic acid in a sample by nucleic acid separation and enrichment by using a bidirectional electro-hydrodynamic flow in a non-newtonian liquid medium, where the method further comprises the addition of probes intended to hybridize with a target nucleic acid so as to introduce a modification of the molecular weight of the target nucleic acid during hybridization with the probes in order to allow the separation of nucleic acid/probe complexes from the nucleic acid alone or the probes alone when the sample is subject to hydrodynamic and electrical action in order to allow the detection of the target nucleic acid.

The technology for nucleic acid separation and enrichment using a bidirectional electro-hydrodynamic flow in a non-newtonian liquid medium is described in particular in the article by Ranchon et al.: “DNA separation and enrichment using electro-hydrodynamic bidirectional flows in viscoelastic liquids.”

The authors have in fact demonstrated that the nucleic acid separation and enrichment operations can be done by simultaneously using spatial modulations of electrical and flow fields via a constriction in a microfluidic channel or capillary.

The technology involves the application of an electric field and a hydrodynamic flow in a non-newtonian liquid medium contained in a channel. These forces increase the molecular weight of the nucleic acids and thus induce a progressive reduction of the migration speed of the nucleic acids, leading to a separation by size in a channel. The channel comprises a constriction or funnel, serving to spatially modulate the hydrostatic and electrical fields so as to stop the displacement of the nucleic acids in a predetermined area and to concentrate the nucleic acids in this set position where they accumulate in a way depending on their molecular weight.

This method of concentration and separation by size is also described in the patent application WO 2016/016470.

Thus, “constriction” is understood in the meaning of the invention as any cross-section variation serving to spatially modulate the hydrostatic and electrical fields so as to stop and concentrate the nucleic acids.

For a capillary, the constriction consists of a reduction of the diameter and means the ratio of the radii before and after the junction zone. Such constrictions are described on the following site: https://picometrics.com/product/biabooster/.

For a constant depth planar microfluidic system, it is the ratio of the widths at the beginning and end of the constriction. Such constrictions are described in the article by Ranchon et al., and also in the application WO 2016/016470.

Examples of constrictions are shown in FIG. 2 and the incorporation thereof in a microfluidic system is shown in FIG. 6. These examples are detailed below.

Other examples of constrictions, where the width and height of the channel are simultaneously modulated, are also described in the article by Lettieri et al.: “A novel microfluidic concept for bioanalysis using freely moving beads trapped in recirculating flows.”

The preferred hydrodynamic flow profiles (characterized notably by given values of flow rate and average speed) are obtained by acting on the pressure control means, so as to generate a pressure difference between the entrance and exit of the channel. In combination, an electric field is generated in the channel by means of electrodes. This electric field is suited for applying an electrostatic force on the nucleic acids which tends to displace them in a direction opposed to the applied hydrodynamic flow.

The applied electric field is from 10 V/m to 10,000 V/m, preferably from 100 V/m to 5000 V/m and more specifically preferred from 200 V/m to 1000 V/m; and/or, the hydrodynamic flow is characterized by an average speed of 1 to 10,000 μm/s, preferably from 5 to 5000 μm/s and more specifically preferred from 10 to 1000 μm/s.

Preferably the liquid is non-newtonian. In the present description, “newtonian fluid” is understood to mean a fluid for which there exists a linear relation between the imposed mechanical stress (force exerted on the fluid per unit surface area) and the shearing of the fluid (meeting the velocity gradient of the fluid). “Non-newtonian fluid” is therefore a fluid which is not a newtonian fluid.

For example, a non-newtonian fluid according to the invention can have a shearing dependent viscosity coefficient; or it can have an elastic behavior. According to an embodiment, the fluid is viscoelastic.

Advantageously, and in the method according to the invention, the hybridization of the probe and the target nucleic acid adds a modification of the molecular weight, by increasing the molecular weight of the target nucleic acid. This molecular weight increase serves to discriminate the target nucleic acid/probe complex from the free probe or from free target nucleic acid subject to a hydrodynamic and electrical action. The modification of the molecular weight of the probe during hybridization with the target serves to selectively enrich the signal relative to the background noise of the solution.

In fact, the nucleic acid/probe complex, depending on the molecular weight thereof, has a response in the flow such that there is a stopping point where the hydrodynamic velocity thereof is compensated by the electrophoretic velocity.

Thus, the probe, when it is not complexed, does not stop in the same area as the nucleic acid/probe complex then allowing detection of the nucleic acid of interest and overcoming the background noise.

The present invention therefore proposes the possibility of selectively enriching the target nucleic acid and eliminating the background noise associated with the probe, and also other nonspecific nucleic acids in order to detect nucleic acids without steps of either attachment or washing.

Thus, the method according to the present invention serves to achieve better sensitivities for nucleic acid detection.

### DESCRIPTION OF THE DRAWINGS

Other specific advantages, goals and features of the present invention will emerge from the following description given, for explanatory purposes and in no way limiting, in light of the attached drawings, wherein:

FIG. 1: Schematic representation of the hydrodynamic and electrophoretic force fields in the channel comprising a constriction allowing the detection of a target nucleic acid;

FIG. 2: Schematic representation of two constrictions;

FIG. 2A: Constriction with a linear geometry;

FIG. 2B: Constriction with a linear geometry;

FIG. 3: Top view schematic representation of the geometry of two channels each comprising a constriction, one with linear geometry, the other with linear geometry where these two constrictions are opposite each other;

FIG. 4: Graphic representation of viscosity as a function of polyvinylpyrrolidone (PVP) concentration data;

FIG. 5: Graphic representation of viscosity as a function of polyvinylpyrrolidone PVP or polyethylene glycol (PEG) concentration data;

FIG. 6: Schematic representation of a microfluidic chip having two channels, each channel comprising two constrictions opposite each other;

FIG. 7A: Sequence of the KRAS target nucleic acid;

FIG. 7B: Structure of the KRAS target nucleic acid;

FIG. 8A: Sequence of the molecular beacon intended to hybridize with the KRAS nucleic acid;

FIG. 8B: Structure of the molecular beacon intended to hybridize with the KRAS nucleic acid;

FIG. 9A: Sequence of the miR21 target nucleic acid;

FIG. 9 B: Structure of the miR21 target nucleic acid;

FIG. 10A: Sequence of the molecular beacon intended to hybridize with the miR21 nucleic acid;

FIG. 10B: Structure of the molecular beacon intended to hybridize with the miR21 nucleic acid;

FIG. 11: Representation of the constriction of the 2.5D microfluidic chip.

### DETAILED DESCRIPTION OF THE INVENTION

Thus, the object of the present invention is to propose a method for detection of a nucleic acid in a sample by separation and enrichment of nucleic acids by using a bidirectional electro-hydrodynamic flow in a non-newtonian liquid medium, said method comprising:
inserting said sample and a probe intended to hybridize with a nucleic acid to form a nucleic acid/probe complex into a channel, where said channel comprises an axis of flow and at least one constriction;
applying a hydrodynamic flow in said channel in combination with the application of an electric field in the channel serving to displace the nucleic acids in the channel along the axis of flow and of stopping and concentrating the nucleic acid/probe complex in an area upstream of said constriction in order to detect said complex.

Alternatively, a mixture comprising a sample and a probe will be prepared and inserted into the channel.

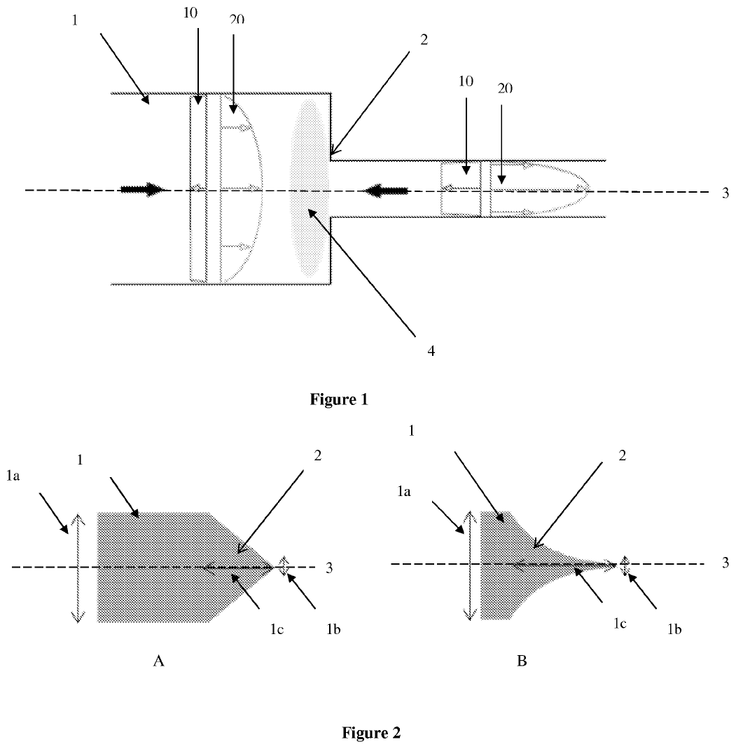

FIG. 1 is a schematic representation of the hydrodynamic and electrophoretic force fields in the channel 1 comprising a constriction 2.

The primary axis of the cylinder is the flow axis 3 in the channel 1.

The nucleic acid/probe complex, depending on the molecular weight thereof, responds in the electro-hydrodynamic flow such that for each flow velocity 20, V₀, there is an electrophoretic velocity, 10, V_{E} to cause it to stop. In other words, if the pair (V₀, V_{E}) is applied, the velocity of the complex is zero.

In fact, with a constriction, a hydrodynamic and electrophoretic speed range is swept spatially: the flow of the two magnitudes is maintained. This configuration serves to define an area 4 where the velocity of the complex is zero. The nucleic acid moves forward towards the constriction on the upstream side and drops back on the downstream side, as shown in FIG. 1. The hybridization of the probe with the target nucleic acid causes a change of the molecular weight of the target nucleic acid and therefore serves to discriminate the target nucleic acid/probe complex from the free probe or free nucleic acids allowing detection thereof.

Here, “nucleic acid detection” is understood to mean the direct or indirect determination of the presence or absence of a specific nucleic acid sequence, including but without limitation to it, detection of a specific sequence in a nucleic acid molecule or the detection of a difference between the sequences of two different nucleic acid molecules, or the detection of a mutation on one nucleic acid.

In the meaning of the present invention, “nucleic acid” is understood to mean single or double-strand DNA or RNA molecules.

Typically, the probe is an oligonucleotide probe, intended to hybridize specifically with the nucleic acid to be detected.

Alternatively, the probe can be selected from an LNA (locked nucleic acid) or PNA (peptide nucleic acid) type synthetic nucleic acids based probe.

Advantageously, these probes have the property of increasing hybridization energy and can as necessary improve the selectivity of the detection.

Typically, the probe comprises a single-strand DNA or RNA sequence that is complementary and anti-parallel to the fragment to be detected, preferably marked.

The marking can be done with a radioisotope or by fluorescence. Alternatively, the probe can comprise an electrochemical probe.

Preferably, a fluorophore will be grafted onto the oligonucleotide probe.

Typically, the fluorophore can be chosen among FAM 6-carboxy-fluorescein, HEX™, JOE™, VIC®, CAL Fluor® Orange 560, Cy3, TetramethylRhodamine, Texas Red®, Cy5, Alexa series, and Atto series.

Advantageously, the method according to the invention allows detecting target nucleic acids with marked linear probes, unsuited until then for detection in solution because of the background noise.

Alternatively, the probe can be a molecular beacon. Molecular beacon is understood to mean a probe formed of a hairpin DNA strand, with each of the ends bearing a fluorophore. One is called reporting and the other quenching (extinguishing).

Typically, the quencher or extinguisher can be selected from Black Hole Quencher®, such as BHQ-1, BHQ-2, BHQ-3, QXL® quenchers, DDQ-I, Dabcyl, Iowa Black® (FQ or RQ), and QSY® 21.

Typically, the fluorophore can be chosen among FAM 6-carboxy-fluorescein, HEX™, JOE™, VIC®, CAL Fluor® Orange 560, Cy3, TetramethylRhodamine, Texas Red®, Cy5, Alexa series, and Atto series.

The sequence comprised in the oligonucleotide probe will be determined depending on the target nucleic acid to be detected.

In one embodiment, the oligonucleotide probe comprises a sequence of size included between 30 and 120 bases.

These probes will be used when the detection of the nucleic acid must be specific.

In another embodiment, the oligonucleotide probe comprises a sequence of size included between 15 and 30 bases.

In this embodiment, the probes will be used to detect one mutation. Preferably, the oligonucleotide probe will have a size of 15 bases.

According to another embodiment, and for the detection of a mutation, the microfluidic system near the constriction can further be heated to a temperature included between 40 and 70° C.; this is done to improve the detection of a mutation.

In fact, a mutation causes a decrease of the hybridization temperature Tₘ.

In the same way, methyl DNA can be detected by heating the microfluidic system near the constriction.

In fact, DNA containing methylated cytosines has different structural and thermodynamic properties (Karberg et al. “A method for quantifying DNA methylation percentage without chemical modification”).

According to another embodiment, the probe further comprises polymers and/or nanoparticles, where said polymers and/or nanoparticles are grafted onto said probe.

Grafting of polymer chains or nanoparticles serves to increase the molecular weight of the probe and does so in order to amplify the molecular weight difference of the target/probe complex and to improve detection.

Typically, the polymer can be a polyethylene glycol (PEG).

In an embodiment, the nanoparticles can be gold nanoparticles grafted to the probe by thiol grafting or polystyrene type polymer nanoparticles grafted to the probe by a biotin-streptavidin bond.

Alternatively, the molecular weight of the probe can be increased by adding a biological molecule selected from a protein and an antibody.

For illustration, specific antibodies of a double-strand DNA could be added after hybridization so as to significantly increase the molecular weight of the probe.

In another embodiment, the probes could be designed by DNA engineering so as to induce a chain reaction associated with the formation of a nucleic acid/probe complex: the complex induces the recruitment of another DNA probe, which serves to amplify the molecular weight difference with the three-body complex.

In an embodiment, this engineering can use the technique of DNA origami by which the interaction of the target and the probe releases some free bases which serve to trigger the attachment of an additional single-strand DNA (amplification effect).

DNA origami is for example described in the article by Wang et al.: “The beauty and Utility of DNA Origami.”

According to another preferred embodiment, the weight of the target nucleic acid/probe complex will have a weight included between 10 kDa and 10⁶ kDa preferably between 20 kDa and 10⁴ kDa and even more preferably between 50 kDa and 10³ kDa.

In another embodiment, the weight of the nucleic acid/probe complex is greater than or equal to 1.5 times the molecular weight of the probe, preferably greater than or equal to 2 times the molecular weight of the probe, and even more preferably, greater than or equal to 4 times the molecular weight of the probe.

In an embodiment, the channel comprises at least one constriction, where said constriction is formed by a first section of the channel with a width 1 and the second section of the channel with a width 1′, where the width 1′ of said second section of the channel is strictly less than the width 1 of said first section of the channel and corresponds to the width of the constriction.

In an embodiment, the ratio of the width 1 of the first section of the channel over the width 1′ of the second section of the channel is greater than 5, preferably greater than 10 or at least greater than 20, or at least greater than 50 and even more preferably greater than 80.

Typically, the width 1 of the first section of the channel will be included between 200 μm and 5000 μm, preferably between 600 μm and 2000 μm.

Preferably, the width 1 of the first section of the channel will be about 800 μm.

Typically, the width 1′ of the second section of the channel will be included between 2 μm and 100 μm, preferably between 5 and 50 μm, and even more preferably, between 5 and 10 μm.

The walls of the channel near the constriction form an angle relative to the flow axis included between 20 and 90°, preferably 30 to 60°, for a channel having a linear geometry.

The length of each constriction can vary between 500 and 2,000 μm.

The area of the opening of the constriction is included between 10 and 3000 μm², preferably 12 and 500 μm², and also preferred between 2 and 100 μm².

The shape of the constriction can be linear or more complex, for example parabolic or exponential.

According to an embodiment, the height of the detection channel of the constriction is included between 1 and 6 μm, preferably between 2 and 4 μm.

FIG. 2 shows schematically two channels, each having one constriction, the one with linear geometry (FIG. 2A), the other with exponential geometry (FIG. 2B).

In FIG. 2A, the channel 1 has the shape of a hollow cylinder with rectangular section. The primary axis of the cylinder is the flow axis 3 in the channel 1.

Perpendicularly to this flow axis 3, a first transverse section 1a of the channel 1 is defined by a width of about 1000 μm and a second transverse section 1b of the channel 1 by a width of about 5 μm and forming the constriction 2.

The constriction has a length 1c of 550 μm.

Thus, the ratio between the area of large section and the small section defining the constriction of the channel defines a concentration factor of about 200.

FIG. 2B represents a constriction having an exponential geometry. The elements shown in FIG. 2B bearing the same references as those from FIGS. 1 and 2A represent the same objects and are not described again below.

The channel shown in FIG. 2B has a first transverse section 1a with a width of about 800 μm, and a second transverse section 1b of about 20 μm. The length of the constriction 1c is about 800 μm.

Thus, the ratio between the area of large section and the small section defining the constriction of the channel defines a concentration factor of about 40.

This constriction therefore has a “valve” effect allowing low molecular weight objects to pass. It thus serves to stop the molecules in a small volume in order to get a large concentration factor, without being too tightening at the risk of stopping non-hybridized probes under the same conditions.

The height of the channel near the constriction is 2 μm.

The height of the channel near the constriction plays a key role for the signal-to-noise ratio: complexes accumulated at the wall become detectable if the signal that they generate is larger than the signal per volume connected to the probes present in the volume. In other words, as the stopped molecules are pressed against the walls, increasing the height of the channel does not necessarily increase the signal, although it does increase the background noise.

Advantageously, the nucleic acid/target complex is stopped upstream from the constriction to avoid leaks associated with an incomplete trapping.

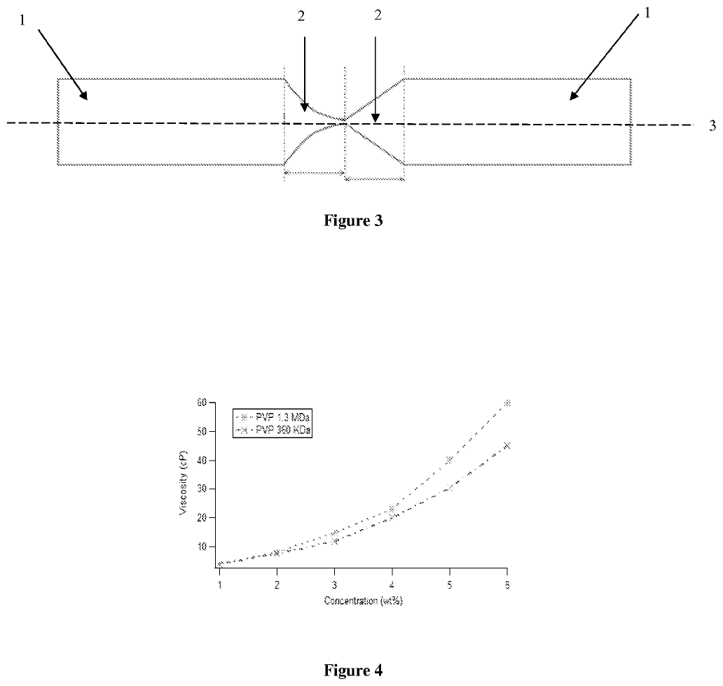

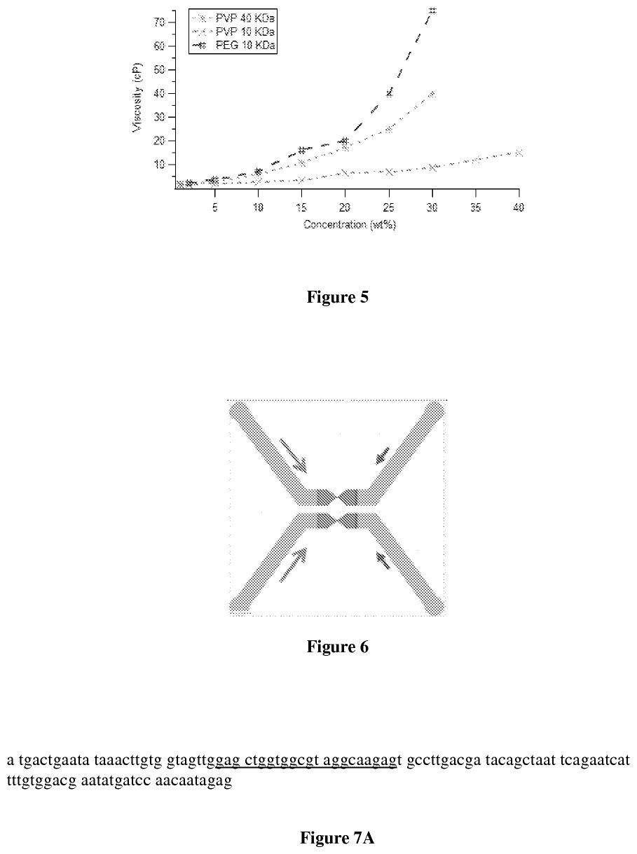

FIG. 3 shows generally two channels 1 each comprising a constriction 2 with a different geometric shape, the constrictions being opposite each other. Typically, the two different constrictions allow comparing side-by-side with a single experimental condition the performance thereof separating, concentrating and detecting nucleic acids.

The channel comprising the constriction can be incorporated in a microfluidic chip.

Various types of microfluidic chips can be used such as microfluidic chips with linear geometry (x shape), microfluidic chips with power law geometry (x^{1.5}, x², x^{2.5}, and x³) and also microfluidic chips with exponential-law geometries (exp(3x), exp(4x), exp(5x) exp(6x) and exp(7x)).

Alternatively, silicon chips fabricated by lithography using grayscale masks can be used (2.5D chip). This fabrication technique is described in the article “Grayscale lithography to fabricate varying-depth nanochannels in a single step” by Naillon, Antoine & Massadi, Hajar & Courson, Rémi & Calmon, Pierre-François & Séveno, Lucie & Prat, Marc & Joseph, Pierre (2016).

As an example, the length of the concentration channel of the chip is 1.7 mm and has a height gradient from 5 μm to 2 μm, and the constriction width is 25 μm. The geometry of the chip is shown in FIG. 11.

Alternatively, the channel can be an opening of a capillary tube. In this case, the constriction corresponds to the junction of two capillaries with different diameters.

The use of capillary tubes can allow an easy multiplexing of channels according to the invention, in the form of bundles of capillary tubes, for example such as those described in the article “Bundled capillary electrophoresis using microstructured fibres” by Rogers et al., Electrophoresis, 32(2):223-229 (2011). In this case, the means for application of the hydrodynamic flow and the means for application of the electrical field can be shared by the set of capillary tubes, or in contrast can be distinct for each of the capillary tubes.

According to an embodiment, the liquid medium has a null shearing viscosity included between 3 cP (centiPoise) and 40 cP, preferably between 10 and 25 cP at ambient temperature.

Typically, two methods can be used to measure the sharing viscosity. It can be measured by dynamic light scattering (DLS) which serves to measure the hydrodynamic radius of colloidal solutions.

DLS measurement can be done with a Malvern ZetaSizer type device. It involves using nanoparticles of a given size R₀ and measuring their apparent hydrodynamic size Rₐ in the solution of undetermined viscosity. The viscosity is given by the ratio Rₐ/R₀.

The elasticity parameters can be measured by using fluorescent nanoparticles of calibrated size R₀ of order 200 nm and the measurement of the spatial fluctuations thereof at ambient temperature by fluorescence video microscopy. The mean squared displacement (MSD) is measured as a function of time τ. These data are adapted according to the model described in (Zanten, P R E, 2000). The following is the analytic expression:

$$MSD ( τ ) = \frac{6 kT}{m} \{ ɛτ + ɛ ( ɛ - λ ) \times [ {e}^{- \frac{t}{2 λ}} cos \{ \frac{\sqrt{4 \frac{λ}{ɛ} - 1}}{2 λ} t \} - 1 ] + \frac{{ɛ}^{2}}{4} {e}^{- \frac{t}{2 λ}} sin \{ \frac{\sqrt{4 \frac{λ}{ɛ} - 1}}{2 λ} t \} \times [ \frac{1}{\sqrt{4 \frac{λ}{ɛ} - 1}} - 3 \sqrt{4 \frac{λ}{ɛ} - 1} ] \}$$

where kT is the thermal kinetic energy, m the mass of the particle, ε=m/6πμR₀ with μ the viscosity of the fluid, λ the relaxation time of the fluid which is μ/E where E is the elasticity of the fluid.

According to an embodiment, the liquid medium comprises uncharged polymers.

The use of a suitable dissolve polymer matrix allows the specific stopping of nucleic acid/probe complexes of interest, and particularly those of small molecular weight for given channel design and for action parameters limited by the materials used.

The term “uncharged” means that the polymers in question have an essentially zero net electrostatic charge in the aforementioned liquid medium. The presence of such polymers for example in an aqueous solution serves to make the liquid medium non-newtonian (for example viscoelastic).

According to an embodiment, the liquid medium comprises uncharged polymers, preferably chosen among polyvinylpyrrolidone (PVP), poly(ethylene glycol), polyacrylamide and/or mixtures thereof.

As an illustration, the liquid medium comprises a PVP and PEG mixture.

Preferably, the uncharged polymers will be selected from polyvinylpyrrolidone 1.3 MDa (PVP 1.3 MDa), polyvinylpyrrolidone 360 KDa (PVP 360 Kda), polyvinylpyrrolidone 40 kDa (PVP 40 kDa), polyvinylpyrrolidone 10 kDa (PVP 10 KDa) and poly(ethylene glycol) 10 KDa (PEG 10 KDa).

In general, high PVP concentrations are favorable for stopping small molecules, but the viscosity becomes large which makes it necessary to impose very high pressures that are not necessarily accessible in commercial equipment. Similarly the electric field cannot be modulated infinitely, because the risks of breakdown are high with any type of device.

According to an embodiment, the uncharged polymers are present at a concentration by mass of 0.5 to 30%, preferably from 2 to 25%, and still more preferably from 3 to 20%.

FIGS. 3 and 4 represent viscosity data as a function of PVP or PEG concentration.

Typically, for detection of low molecular weight nucleic acids (under 100 base pairs), high viscosity solutions will be used.

For example, the liquid medium comprises PVP 40 kDa in a concentration by mass of order 18% or PVP 1.3 MDa with a concentration by mass of order 3%.

Separation is easier for DNA in the 100 to 1000 base pair range were good performance is obtained for all conditions.

Separation conditions for high molecular weight nucleic acids, of order 10,000 base pairs or more, are obtained with more dilute solutions. For illustration, a liquid medium comprising PVP 2.5 kDa in a concentration by weight of order 2% can be used.

The person skilled in the art will advantageously know to select the polymer and concentration thereof according to the target to be detected.

According to a preferred variant of the method according to the invention, the applied electric field is from 0.1 kV/m to 10³ kV/m, preferably from 1 kV/m to 500 kV/m and more specifically preferred from 100 kV/m to 200 kV/m; and/or, the hydrodynamic flow is characterized by an average speed of 0.1 to 10³ mm/s, preferably from 1 to 100 mm/s and even more preferred from 5 to 10 mm/s.

The preferred hydrodynamic flow profiles (characterized notably by given values of flow rate and average speed) are obtained by acting on the pressure control means, so as to generate a pressure difference between the entrance and exit of the channel. For example, the preferred hydrodynamic flow profiles can be obtained with a voltage difference less than 12 bars, preferably included between 50 mbar to 10 bars, preferably between 2 and 6 bars and more specifically preferred between 0.1 to 3 bars.

In combination, an electric field is generated in the channel by means of electrodes. This electric field is suited for applying an electrostatic force on the electrically charged objects which tends to displace them in a direction opposed to the applied hydrodynamic flow.

Typically, the voltage will be under 400 V and preferably included between 10 to 300 V and more specifically preferred from 100 to 200 V.

In an embodiment, the sample is inserted in an insertion area of the channel and the displacement of the electrically charged objects is done from the insertion area towards a detection area of the channel, the method further comprising:
the detection of the nucleic acid/probe complex arriving in a detection area.

Typically, the detection can be done near the constriction.

In another embodiment, the detection can be done downstream from the constriction.

The concentration factor depends on time, thus the detection time is included between 10 and 5000 seconds, preferably between 50 and 1000 seconds, and preferably between 100 and 500 seconds, and this is done to reach a sufficient enrichment.

The person skilled in the art will know how to determine the analysis time while also considering the dissociation time of the nucleic acid/probe complex.

### EXAMPLES

In examples 1 to 5 which follow, the following materials and methods will be used:
Microfluidic chip

In the following examples, one microfluidic chip with two channels manipulated with identical parameters for action were used. This microfluidic chip is shown in FIG. 6 and comprises two channels each comprising two constrictions placed facing such as shown in FIG. 3.

This microfluidic chip is also described in Malbec et al.

This chip was used experimentally. Similar results can be obtained with a channel or capillary comprising a constriction.

This system was used to simultaneously evaluate the signal in a channel where a sample was inserted in the top channel in FIG. 6 and a sample in the bottom channel in FIG. 6, the top channel serving as control. The two channels were observed simultaneously by fluorescence video microscopy.
Analysis

The videos were analyzed with ImageJ, a program allowing tracing the fluorescence intensities and also determining the position of the nucleic acids in the channel.

The intensities are adjusted according to a Gaussian distribution and the resolution is calculated according to the ratio of the distance between consecutive Gaussian shaped peaks and the sum of the height thereof.

### Example 1: Detection of the KRAS Proto-Oncogene

In this example, the invention is used to separate, concentrate and detect the KRAS proto-oncogene.

Thus, the target nucleic acid is the KRAS proto-oncogene comprising 111 bases (SEQ ID N^{o} 1).

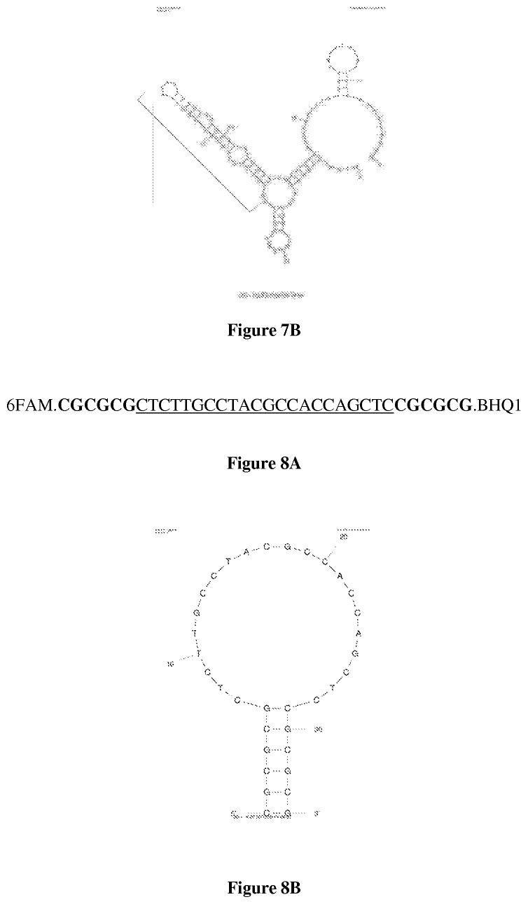

The sequence thereof is shown in FIG. 7A, and the structure thereof is as shown in FIG. 7B.

The associated probe is a molecular beacon and corresponds to the KRAS probe, comprising 32 bases (SEQ ID N^{o} 2) together with a fluorophore: 6 FAM, and a quencher: Black Hole Quencher®: BHQ1.

The sequence thereof is shown in FIG. 8A, and the structure thereof is as shown in FIG. 8B.

The area of interaction of the KRAS target nucleic acid with the probe is designated by the bracket on FIG. 7B.

The samples are diluted in a separation buffer comprising TBE 1× and PVP 1.3 MDa with a concentration by mass of about 5%.

The viscosity of the non-newtonian liquid medium is about 3 cP at ambient temperature.

A sample comprising 100 nmol of probe was inserted in the top channel and a sample comprising 100 nmol of probe incubated for about one hour at 40° C. with 1 μmole of target nucleic acid was inserted in the bottom channel.

A total pressure difference of 1.7 bars and a voltage difference of 168 V were used with crossed hydrodynamic and electrophoretic flows, meaning oriented along the flow axis but along opposite axes. With this pressure/voltage pair, the enrichment and therefore the detection of the complex of KRAS nucleic acid and its probe was possible.

This voltage/pressure pair leads to a hydrodynamic velocity of the fluid of 2 cm/s and an electric field value present within the constriction of 700 kV/m.

The pressure and voltage parameters were modulated. A total pressure difference of 1.7 bars and a voltage difference of 312 V were used with crossed hydrodynamic and electrophoretic flows. This pressure/voltage pair serve to enrich the probe and detect only it.

This voltage/pressure pair leads to a hydrodynamic velocity of the fluid of 2 cm/s and an electric field value present within the constriction of 1300 kV/m.

The method according to the invention thus serves to detect the nucleic acid/probe complex at a given pressure and voltage pair, and to eliminate the background noise associated with the probe.

### Example 2: Detection of miR 21

In this example, the invention is used to separate, concentrate and detect miR 21.

Thus, the target nucleic acid is the miR21 comprising 22 bases (SEQ ID N^{o} 3).

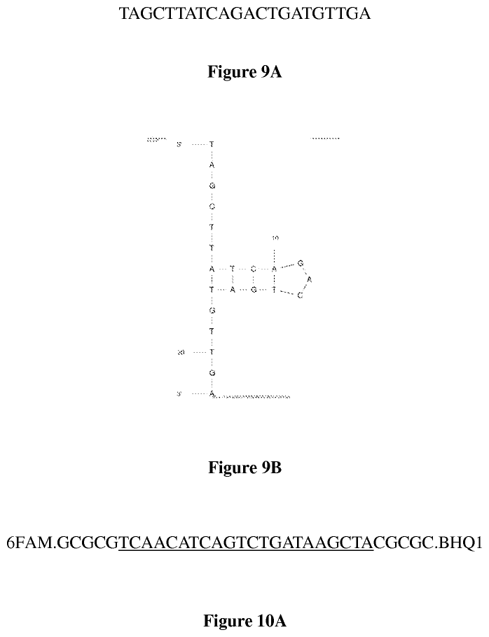

The sequence thereof is shown in FIG. 9 A, and the structure thereof is as shown in FIG. 9 B.

The associated probe is the miR21 probe, comprising 32 bases (SEQ ID N^{o} 4) together with a fluorophore: 6 FAM, and a quencher: Black Hole Quencher®: BHQ1.

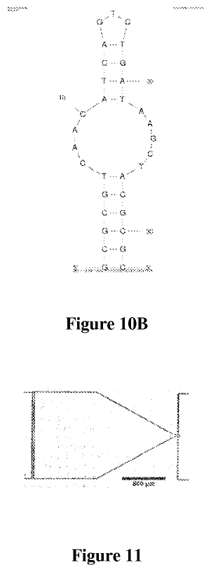

The sequence thereof is shown in FIG. 10 A, and the structure thereof is as shown in FIG. 10 B.

The samples are diluted in a separation buffer comprising TBE 1× and PVP 1.3 MDa with a concentration by mass of about 5%.

The viscosity of the non-newtonian liquid medium is about 3 cP at ambient temperature.

100 nmol of miR 21 probe and 300 nmol of miR 21 nucleic acid were used.

A sample comprising the probe was inserted in the top channel and a sample comprising the probe and target nucleic acid was inserted in the bottom channel.

A total pressure difference of 2 bars and a voltage difference of 235 V were used with crossed hydrodynamic and electrophoretic flows, meaning oriented along the flow axis but along opposite axes.

With this pressure/voltage pair, the enrichment and therefore the detection of the complex of miR21 nucleic acid and probe therefor was possible.

The pressure/voltage pair was modulated and a total pressure difference of 2 bars and a voltage difference of 314 V were used with crossed hydrodynamic and electrophoretic flows. Under these conditions, the probe alone was concentrated and therefore detected.

The method according to the invention thus serves to detect the nucleic acid/probe complex at a given pressure and voltage pair, and to eliminate the background noise associated with the probe.

### Example 3: Detection of Nucleic Acid by Using a Straight Linear Probe in Excess

In this example, the invention is used to separate, concentrate and detect the following nucleic acid:

(SEQ ID No 5)
5′AAAAAAAAAAAAAAAAAAAAAAAAAAAAAAAAAATAGCTTATCAGAC
TGATGTTGAAAAAAAAAAAAAAAAAAAAAAAAAAAAAAAAAAAA-3′

The following is the probe: 5′-6FAM-TCAACATCAGTCTGATAAGCTA-3′ (SEQ ID N^{o} 6)

The samples are diluted in the separation buffer TBE 1×, PVP 5% 1.3 MDa.

The viscosity of the non-newtonian liquid medium is about 3 cP at ambient temperature.

A sample comprising 100 nmol of probe and 1 nmol of target was injected into the top channel of the microfluidic chip.

A sample comprising 100 nmol of probe and 10 nmol of target was injected into the bottom channel.

A pressure difference of 2 bars and a voltage of 230 V were used with crossed hydrodynamic and electrophoretic flows. The method according to the invention was thus able to detect the nucleic acid/target complexes alone for given voltage and pressure parameters.

A pressure difference of 2.5 bars and a voltage of 288 V were used with crossed hydrodynamic and electrophoretic flows. Under these conditions, only the probe can be detected.

Advantageously, the method according to the invention serves to detect low concentrations of nucleic acids.

It is additionally possible to saturate the solution with probe and to effectively detect the target nucleic acid because the method according to the invention serves to enrich the signal and reduce the background noise.

### Example 4: Detection of a Target Sequence in a Sample Comprising Circulating DNA

1) Sample Preparation

4 mL of blood plasmin was prepared with a norgenbiotek Plasma/Serum Circulating DNA Purification Midi Kit.

Purified DNA was eluted in 50 μL of TE 1×.

50 μL of purified DNA was diluted with 450 μL of TBE 1× and PVP 1.3M 5%, and then were filtered on the surface filter comprising 0.22 μm pores.

2) Preparation of Target Nucleic Acids and Probes

The following is the target nucleic acid:

(SEQ ID No 7)
5′AAAAAAAAAAAAAAAAAAAAAAAAAAAAAAAAAAATAGCTTATCAGA
CTGATGTTGAAAAAAAAAAAAAAAAAAAAAAAAAAAAAAAAAAAA-3′

The probe used comprises 22 bases, the fluorophore: 6 FAM and is the following: 5′-6FAM-TCAACATCAGTCTGATAAGCTA-3′ (SEQ ID N^{o} 8).

A sample comprising 100 nmol of probe was injected into the top channel of the microfluidic chip.

A sample comprising 10 nmol of nucleic acid to be detected and also 100 nmol of probe was injected into the bottom channel.

A pressure difference of 1.5 bar and a voltage difference of 280 V were used with crossed hydrodynamic and electrophoretic flows, meaning oriented along the flow axis but along opposite axes.

This voltage/pressure pair leads to a hydrodynamic velocity of the fluid of 1.8 cm/s and an electric field value present within the constriction of 1170 kV/m.

At these pressure and voltage differences, the probes alone were stopped in an area upstream of the constriction.

The pressure difference was changed and a pressure difference of 1.75 bar and a difference of 280 V were used with crossed hydrodynamic and electrophoretic fluxes such as previously mentioned.

This voltage/pressure pair leads to a hydrodynamic velocity of the fluid of 2.1 cm/s and an electric field value present within the constriction of 1170 kV/m.

This pressure and voltage pair serves to stop the target nucleic acid/probe complex to be detected and to overcome the background noise formed by the single probes, since they were not stopped by these conditions.

The method according the invention thus allowed detecting target nucleic acids at low concentrations in complex samples.

Thus, and in the previous examples, for a given pressure, it is possible to impose a weak electric field such that only the nucleic acid/probe complexes are stopped, whereas the probes circulate freely through the constriction. By increasing the electric field, the nucleic acid/probe complexes migrate backward. It is thus possible, at a given threshold to observe an accumulation of the signal giving evidence to the detection of the nucleic acid/probe complex, and thus, independently isolate the nucleic acid/probe complexes.

Thus, by correctly adjusting the voltage and pressure parameters, and the constriction, it is possible to selectively enrich the nucleic acid/probe complex and to eliminate the background noise associated with the probe.

The method according to the invention therefore serves to reach better sensitivity levels than those obtained in volume measurement.

### Example 5: Selective Enrichment of the Probe/Target Nucleic Acid Complex Versus Free Probe

The target nucleic acid is a single-strand DNA sequence with 22 bases and the probe is a probe having a complementary sequence, marked by a 6-FAM fluorophore.

A sample comprising 1 μmol of probe and 1 μmol of target nucleic acid was used.

The voltage/pressure pair was adjusted in order to allow the formation of the nucleic acid/target complex.

The target nucleic acid complex was detected after 1 second. After 10 seconds of enrichment according to the voltage/pressure parameters, all the nucleic acid/target complexes are stopped upstream from the constriction.

The experiment was done by using 1 nmol of probe and 1 nmol of target nucleic acid.

Although the signal was weaker, it is possible to detect, with the method according to the invention, the target nucleic acid/probe complex after 10 seconds of enrichment according to the pressure/voltage parameters.

The enrichment factor is calculated by measuring the intensity of the fluorescence near the constriction as a function of the time applied for the enrichment.

Similar enrichment factors were obtained for each of the experiments after 9 seconds. Advantageously, an enrichment factor of 160 was obtained for the experiment done on samples comprising 1 μmol of probe and 1 μmol of target nucleic acids, and an enrichment factor of 143 for the experiment done on samples comprising 1 nmol of probe and 1 nmol of target nucleic acid.

Thus, the method according to the invention advantageously serves to enrich selectively nucleic acids at low concentration in order to allow their detection.

### Example 6: Detection Threshold

The target nucleic acid is a single-strand DNA sequence with 99 bases and the probe is a probe having a complementary sequence of 34 bases marked by a 6-FAM fluorophore (6-carboxyfluorescéine) (Eurogentec, Liège, Belgium).

Thus, the following is the target nucleic acid:

(SEQ ID No 9)
5′-TTTTTTTTTTTTTTTTTTTTTTTTTTTTTTTTTAAATGCTGGGCGA
TAAGAGGTTCCGTGTAGTCAATTTTTTTTTTTTTTTTTTTTTTTTTTTT
TTTT-3′

The probe used is the following: 5′-6FAM-TTGACTACACGGAACCTCTAATCGCCCAGCATTT-3′ (SEQ ID N^{o} 10).

In this example, 2 μmol of probe is mixed with various concentrations of target nucleic acids from 2 pmol to 2 μmol in PBS 0.2x.

The resulting solutions were heated at 92° C. for two minutes and then slowly cooled to ambient temperature.

20 μL of the probe/target nucleic acid mixture was diluted in 980 μL of freshly prepared buffer and filtered at 0.2 μm containing 5% PVP 1.3 MDa in PBS 0.2x.

The resulting final solutions contain constant concentrations of probes (20 nmol) and variable concentrations of target nucleic acids (from 20 fmol to 20 nmol).

For the enrichment and detection of the nucleic acid/probe complex, silicone chips fabricated by lithography using grayscale level masks were used.

The length of the concentration channel of the chip is 1.7 mm and has a height gradient from 5 μm to 2 μm.

The constriction width was set at 25 μm. The geometry of the chip is shown in FIG. 11.

The approach channel was filled with ethanol to eliminate air bubbles. There was next carefully rinsed with working buffer before inserting 40 μL a solution containing various ratios of concentration of target nucleic acid to probes (constant concentration of probe (20 nmol) and variable concentrations of target nucleic acids (from 20 fmol to 20 nmol).

The viscosity of the non-newtonian liquid medium is about 31 cP at ambient temperature.

A total pressure difference of 5 bars and a voltage difference of 190 V were used with crossed hydrodynamic and electrophoretic flows. This pressure/voltage pair served to selectively enrich the nucleic acid/probe complex and therefore the detection thereof.

This voltage/pressure pair leads to a hydrodynamic velocity of the fluid of 10.18 cm/s and an electric field value present within the constriction of 100 kV/m.

The method according to the invention thus serves to detect the nucleic acid/probe complex at a given pressure and voltage pair, and to eliminate the background noise associated with the probe and also has high sensitivity in that the method according to the invention advantageously allows detecting the target nucleic acid at a concentration of 20 fmol.

1. A method for detection of a nucleic acid in a sample by separation and enrichment of nucleic acids by using a bidirectional electro-hydrodynamic flow in a non-newtonian liquid medium, said method comprising:
inserting said sample and a probe intended to hybridize with a nucleic acid to form a nucleic acid/probe complex into a channel, where said channel comprises an axis of flow and at least one constriction;
applying a hydrodynamic flow in said channel in combination with the application of an electric field in the channel serving to displace the nucleic acids in the channel along the axis of flow and to stop and concentrate the nucleic acid/probe complex in an area upstream of said constriction in order to detect said complex.

2. The method according to claim 1 wherein the probe is an oligonucleotide probe.

3. The method according to claim 2 wherein the oligonucleotide probe comprises from 30 to 120 bases.

4. The method according to claim 2 wherein the oligonucleotide probe comprises from 15 to 30 bases.

5. The method according to claim 1, wherein said probe further comprises polymers and/or nanoparticles, where said polymers and/or nanoparticles are grafted onto said probe.

6. The method according to claim 1, wherein the molecular weight of the nucleic acid/probe complex is greater than or equal to 2 times the molecular weight of the probe.

7. The method according to claim 1, wherein the viscosity of the non-newtonian liquid medium is from 3 cP to 40 cP, at ambient temperature.

8. The method according to claim 1, wherein said non-newtonian liquid medium comprises uncharged polymers.

9. The method according to claim 1, wherein the detection time is from 10 to 5000 seconds.

10. The method according to claim 1, wherein:
an applied pressure difference is less than 12 bars, and/or
an applied voltage is less than 400 V.

11. The method of claim 6, wherein the molecular weight of the nucleic acid/probe complex is greater than or equal to 4 times the molecular weight of the probe.

12. The method according to claim 7, wherein the viscosity of the non-newtonian liquid medium is from 10 to 25 cP.

13. The method of claim 8, wherein the uncharged polymers comprise polyvinylpyrrolidone, poly(ethylene glycol) and/or mixtures thereof.

14. The method according to claim 9, wherein the detection time is from 50 to 1000 seconds or from 100 to 500 seconds.

15. The method according to claim 10, wherein:
the applied pressure difference is from 50 mbar to 10 bars, and/or
the applied voltage is from 10 to 300 V.

16. The method of claim 15, wherein the applied voltage is from 100 to 200 V.
